(19) United States
(12) Patent Application Publication
(10) Pub. No.: US 2018/0256561 A1
(43) Pub. Date: Sep. 13, 2018

(54) Pharmaceutical Combination

(71) Applicant: GLAXOSMITHKLINE INTELLECTUAL PROPERTY DEVELOPMENT LIMITED, Brentford, Middlesex (GB)

(72) Inventor: Colin John FISH, Ware, Hertfordshire (GB)

(21) Appl. No.: 15/542,125

(22) PCT Filed: Jan. 11, 2016
(86) PCT No.: PCT/EP2016/050370

§ 371 Date: Jul. 7, 2017

(30) Foreign Application Priority Data
Jan. 12, 2015 (GB) 1500447.6

(51) Int. Cl. A61K 31/4709; A61K 31/58; A61K 9/00; A61P 11/00

(52) U.S. Cl. CPC A61K 31/4709; A61P 11/00; A61K 9/0075; A61K 31/58

(57) ABSTRACT

This invention provides a pharmaceutical combination comprising biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester, or a pharmaceutically acceptable salt thereof, and fluticasone furoate, and use of the pharmaceutical combination in therapy, for example in the treatment or prophylaxis of respiratory disorders or diseases, for example pulmonary disorders such as chronic obstructive pulmonary disease (COPD) asthma and Asthma-COPD Overlap Syndrome (ACOS).

### FIELD OF THE INVENTION

This invention provides a pharmaceutical combination comprising biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester, or a pharmaceutically acceptable salt thereof, and fluticasone furoate, and use of the pharmaceutical combination in therapy, for example in the treatment or prophylaxis of respiratory disorders or diseases, for example pulmonary disorders such as chronic obstructive pulmonary disease (COPD), asthma and Asthma-COPD Overlap Syndrome (ACOS). The present invention also provides compositions and medicaments which can be used in the treatment or prophylaxis of respiratory disorders or diseases, for example pulmonary disorders.

### BACKGROUND OF THE INVENTION

Chronic obstructive pulmonary disease (COPD) is a chronic disease characterised by airways obstruction and reduced maximum expiratory flow from the lungs that manifests as persistent daily symptoms, such as shortness of breath (dyspnoea), and limitation of the ability to perform daily activities or exertion. Furthermore, there are periodic exacerbations of the condition that result in worsening of the day-to-day symptoms and activity limitation, and can also lead to hospitalisation of the patient because of the severity of the worsening symptoms/limitation. In addition, there is a progressive decline in lung function (disease progression) over several years.

Bronchodilator treatment in COPD includes, but is not necessarily limited to reducing symptoms, particularly dyspnoea, to allow a patient to undertake more daily activities and other activities that require exertion, and preventing exacerbations.

Asthma is a chronic condition, which is characterised by widespread, variable and reversible airflow obstruction. Symptoms include coughing, wheezing, breathlessness and/or a tight feeling in the chest. Asthma attacks are generally caused by exposure to a trigger, such as pollen, dust or other allergens, which causes constriction of the airways (bronchoconstriction). It will be appreciated that a subject suffering from a condition such as asthma, may variously from time to time display no overt symptoms of the condition, or may suffer from periodic attacks during which symptoms are displayed or may experience exacerbations or worsening of the condition.

Inhaled corticosteroids (ICS) are currently considered the most effective anti-inflammatory treatments for all severities of persistent asthma: Global Initiative for Asthma (GINA), 2011, http://www.ginasthma.org/documents/14. Treatment with ICS controls asthma symptoms, improves quality of life and lung function, decreases airway hyperresponsiveness, controls airway inflammation, and reduces the frequency and severity of asthma exacerbations, thereby reducing asthma mortality. The dose of ICS is selected based on the severity of the patient's asthma. However, to achieve asthma control, add-on therapy with another controller, in particular an inhaled long-acting beta-adrenoceptor agonist (LABA), is often preferred in place of increasing the dose of ICS. Inhaled LABA therapy may, however, be associated with increased risk of serious asthma-related events (including hospitalisation and death) particularly when used as a monotherapy for asthma: GINA, 2011.

Asthma-COPD overlap syndrome (ACOS) is a commonly encountered yet loosely defined clinical entity and is characterised by persistent airflow limitation with several features usually associated with asthma and several features usually associated with COPD. ACOS accounts for approximately 15-25% of the obstructive airway diseases and patients experience worse outcomes compared with asthma or COPD alone. Patients with ACOS have the combined risk factors of smoking and atopy, are generally younger than patients with COPD and experience acute exacerbations with higher frequency and greater severity than lone COPD. ACOS is therefore identified by the features that it shares with both asthma and COPD: “Diagnosis of Diseases of Chronic Airflow Limitation: Asthma, COPD and Asthma-COPD Overlap Syndrome (ACOS)” GINA, 2014: http://www.ginasthma.org/documents/14.

There exists a need for alternative therapies for respiratory disorders and diseases that are able to improve patient compliance or provide improved efficacy over current treatment options.

International Patent application no PCT/US2004/004449, publication no. WO 2004/074246 A2 (Theravance Inc) discloses novel biphenyl compounds that are useful as therapeutic agents for treating pulmonary disorders, such as chronic obstructive pulmonary disease (COPD) and asthma. In particular, the compound biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester is specifically disclosed as possessing both muscarinic antagonist and β₂ adrenergic receptor agonist activity. The chemical structure of biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester is represented by formula (I):

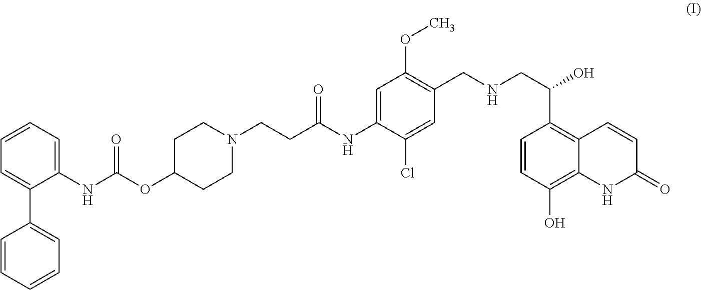 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

International Patent application no PCT/EP2007/051196, publication no. WO 2007/090859 A1 (Glaxo Group Limited) discloses a succinic acid salt of the compound of formula (I), which is stable, non-delicquescent and crystalline, and processes for its manufacture. WO 2007/090859 also discloses that a succinic acid salt of a compound of formula (I) may be used in combination with a steroidal anti-inflammatory agent such as “methyl prednisolone, prednisolone, dexamethasone, fluticasone propionate, 6,9-difluoro-17α-[(2-furanylcarbonyl)oxy]-11-hydroxy-16-methyl-3-oxoandrosta-1,4-diene-17-carbothioic acid S-fluoromethyl ester, 6,9-difluoro-11-hydroxy-16α-methyl-3-oxo-17β-propionyloxy-androsta-1,4-diene-17-carbothioic add S-(2-oxotetrahydrofuran-3S-yl) ester, beclomethasone esters (e.g. the 17-propionate ester or the 17,21-dipropionate ester), budesonide, flunisolide, mometasone esters (e.g. the furoate ester), triamdnolone acetonide, rofleponide, ciclesonide, butixocort propionate, RPR-106541, ST-126 and the like, or pharmaceutically-acceptable salts thereof. In a particular embodiment, the steroidal anti-inflammatory agent is 6α,9α-difluoro-17α-[(2-furanylcarbonyl)oxy]-11β-hydroxy-16α-methyl-3-oxoandrosta-1,4-diene-17β-carbothioic acid S-fluoromethyl ester or a pharmaceutically acceptable salt or solvate thereof” (page 19 line 29-page 20 line 9). 6α,9α-difluoro-17α-[(2-furanylcarbonyl)oxy]-11β-hydroxy-16α-methyl-3-oxoandrosta-1,4-diene-17β-carbothioic acid S-fluoromethyl ester is generically known as fluticasone furoate.

WO2006/023454 discloses a 1,2-ethandisulfonic acid salt (edisylate salt) of the compound of formula (I).
WO2010/119064 discloses a process for the preparation of the compound of formula (I).

### SUMMARY OF THE INVENTION

In a first aspect, the present invention provides a pharmaceutical combination comprising:

a) Compound 1, biphenyl-2-ylcarbamic add 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester

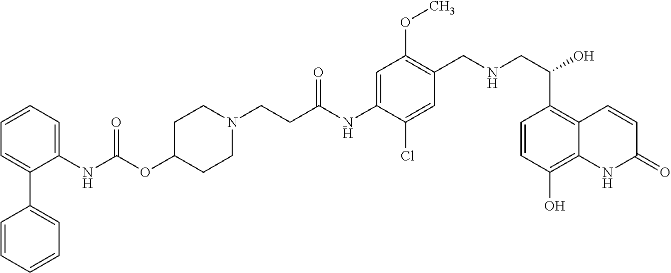 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, which is provided in an amount equivalent to: about 150 mcg to about 300 mcg of the free base of Compound 1
and
b) about 100 mcg to about 200 mcg of Compound 2, fluticasone furoate

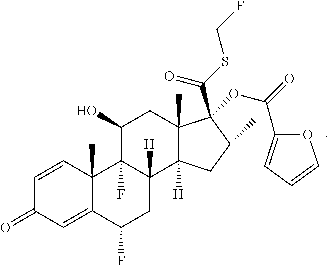

In a second aspect the present invention provides a pharmaceutical combination product comprising:

a) Compound 1, biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester

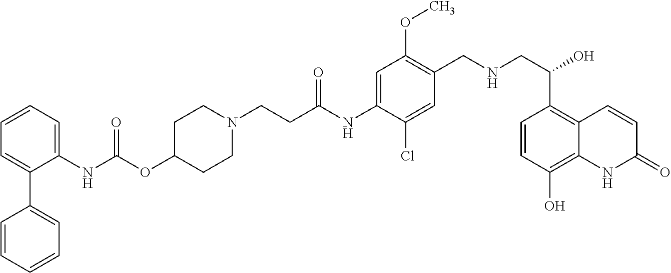 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, wherein the free base of Compound 1 is provided in an amount of 150 mcg to 300 mcg
and
b) 100 mcg to 200 mcg of Compound 2, fluticasone furoate

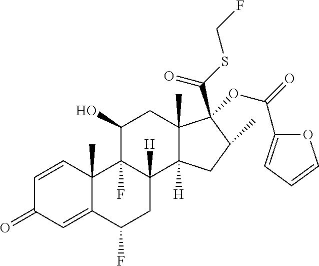

for the treatment of inflammatory or respiratory disorders, such as COPD and asthma.

It is to be understood that references hereinbelow to “Compound 1” (other than references to a free base of Compound 1) are equally applicable to a pharmaceutically acceptable salt of Compound 1.

In one embodiment, the pharmaceutical combination product comprises Compound 1 in the form of a succinate salt. In another embodiment, the succinate salt is the crystalline Form 1 succinate salt as described in WO 2007/090859 A1.

In one embodiment, the pharmaceutical combination product comprises the free base of Compound 1 in an amount of 150 to 300 mcg and Compound 2 in an amount of 100 to 200 mcg.

In one embodiment the 150 mcg to 300 mcg of the free base of Compound 1 is provided in the form of a succinate salt of Compound 1.

In one embodiment Compound 1 and Compound 2 are the sole active ingredients in said pharmaceutical combination product.

This invention also provides for use of the pharmaceutical combination product in the manufacture of a medicament for the treatment or prophylaxis of respiratory disorders or diseases, for example a pulmonary disorder.

In one embodiment the use of the pharmaceutical combination product is for the manufacture of a medicament for the treatment or prophylaxis of respiratory disorders or diseases, by simultaneous or sequential administration of Compound 1 and Compound 2.

In another embodiment the use of the pharmaceutical combination product is for the manufacture of a medicament for the treatment or prophylaxis of COPD asthma and/or ACOS by simultaneous or sequential administration of Compound 1 and Compound 2.

The invention also provides said pharmaceutical combination product for use in therapy, for example in the treatment or prophylaxis of respiratory disorders or diseases, such as COPD, asthma and/or ACOS.

Another embodiment of the invention is a method for the treatment or prophylaxis of respiratory disorders or diseases, for example a pulmonary disorder, comprising administering either simultaneously or sequentially, to a patient in need thereof, a pharmaceutical combination product comprising Compound 1 and Compound 2.

In one embodiment of the invention the respiratory disorder or disease is selected from the group consisting of COPD, chronic bronchitis, asthma, chronic respiratory obstruction, pulmonary emphysema, allergic rhinitis, small airways disease, bronchiectasis, asthma, ACOS and cystic fibrosis. In another embodiment the respiratory disorder or disease is COPD, asthma and/or ACOS. In another embodiment the respiratory disorder or disease is COPD or asthma. In a further embodiment, the respiratory disorder or disease is COPD.

In another embodiment of the invention the pharmaceutical combination product may be used for the treatment or prophylaxis of respiratory disorders or diseases, for example a pulmonary disorder, and more specifically the treatment or prophylaxis of COPD, asthma and/or ACOS, by simultaneous or sequential administration of Compound 1 and Compound 2.

### DETAILED DESCRIPTION OF THE INVENTION

The present invention is directed to a pharmaceutical combination comprising

a) Compound 1, biphenyl-2-ylcarbamic acid 1-[2-(2-chloro-4-{[(R)-2-hydroxy-2-(8-hydroxy-2-oxo-1,2-dihydroquinolin-5-ylethylamino]methyl}-5-methoxyphenylcarbamoyl)ethyl]piperidin-4-yl ester

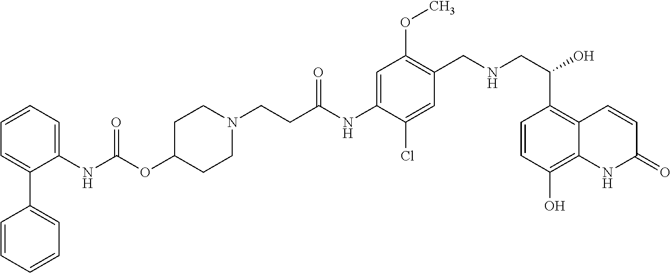 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, which is provided in an amount equivalent to: about 150 mcg to about 300 mcg of the free base of Compound 1
and
b) about 100 mcg to about 200 mcg of Compound 2, fluticasone furoate

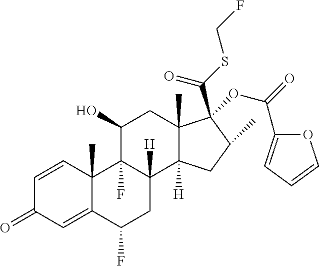

As used herein, “mcg” means micrograms.

The combination of Compounds 1 and 2 is considered to have potential in the treatment or prophylaxis of respiratory disorders or diseases such as COPD, chronic bronchitis, asthma, chronic respiratory obstruction, pulmonary emphysema, allergic rhinitis, small airways disease, bronchiectasis and cystic fibrosis.

As used herein, unless otherwise indicated, “treat”, “treating” or “treatment” in reference to a disease means: (1) to ameliorate the disease or one or more of the biological manifestations of the disease (2) to interfere with (a) one or more points in the biological cascade that leads to or is responsible for the disease or (b) one or more of the biological manifestations of the disease, (3) to alleviate one or more of the symptoms or effects associated with the disease, (4) to slow the progression of the disease or one or more of the biological manifestations of the disease, and/or (5) to diminish the likelihood of severity of a disease or biological manifestations of the disease. For example, for asthma, the term ‘treatment’ is intended to encompass minimisation or prevention of periodic asthma attacks or exacerbations of the existing condition. Such treatment may be referred to as ‘maintenance treatment’ or ‘maintenance therapy’.

As used herein, unless otherwise indicated, “prophylaxis” means the preventative administration of a drug to diminish the likelihood of the onset of or to delay the onset of a disease or biological manifestation thereof. The skilled artisan will appreciate that “prophylaxis” is not an absolute term. In medicine, “prophylaxis” is understood to refer to the prophylactic or preventative administration of a drug to substantially diminish the likelihood or severity of a disorder or biological manifestation thereof, or to delay the onset of such disorder or biological manifestation thereof.

It will be further appreciated by those skilled in the art that references herein to “treatment” refer to the treatment of established conditions. The compounds of Formula (I) and pharmaceutically acceptable salts thereof may, depending on the condition, also be useful in the prophylaxis (prevention) of certain diseases. Thus, in one embodiment, there is provided the treatment or prophylaxis of a disease. In another embodiment, there is provided the treatment of a disease. In a further embodiment, there is provided the prophylaxis of a disease.

In one embodiment, the route of administration is by inhalation via the mouth or nose. In a further embodiment, the route of administration is by inhalation via the mouth.

In one embodiment, Compound 1, or a pharmaceutically acceptable salt thereof, is present in the pharmaceutical combination in an amount equivalent to 150 to 300 mcg of the free base of Compound 1.

In another embodiment, the amount of Compound 1, or a pharmaceutically acceptable salt thereof, is equivalent to about 150 mcg of the free base of Compound 1. In a yet further embodiment, the amount present is equivalent to 150 mcg±50 mcg, or 150 mcg±45 mcg, or 150 mcg±40 mcg, or 150 mcg±35 mcg, or 150 mcg±30 mcg, or 150 mcg±25 mcg, or 150 mcg±20 mcg, or 150 mcg±15 mcg, or 150 mcg±10 mcg, or 150 mcg±5 mcg of the free base of Compound 1. In a yet further embodiment, the amount present is equivalent to an amount selected from 150 mcg, 145 mcg, 140 mcg, 135 mcg, 130 mg, 125 mcg, 120 mcg, 115 mcg, 110 mcg, 105 mcg and 100 mcg.

In yet another embodiment, the amount of Compound 1, or a pharmaceutically acceptable salt thereof, is equivalent to 150 mcg of the free base of Compound 1.

In a further embodiment, the amount of Compound 1, or a pharmaceutically acceptable salt thereof, is equivalent to about 300 mcg of the free base of Compound 1. In a yet further embodiment, the amount present is equivalent to 300 mcg±50 mcg, or 300 mcg±45 mcg, or 300 mcg±40 mcg, or 300 mcg±35 mcg, or 300 mcg±30 mcg, or 300 mcg±25 mcg, or 300 mcg±20 mcg, or 300 mcg±15 mcg, or 300 mcg±10 mcg, or 300 mcg±5 mcg of the free base of Compound 1. In a yet further embodiment, the amount present is equivalent to an amount selected from 300 mcg, 295 mcg, 290 mcg, 285 mcg, 280 mg, 275 mcg, 270 mcg, 265 mcg, 260 mcg, 255 mcg and 250 mcg.

In yet another embodiment, the amount of Compound 1, or a pharmaceutically acceptable salt thereof, is equivalent to 300 mcg of the free base of Compound 1.

In a further embodiment, the amount of Compound 1, or a pharmaceutically acceptable salt thereof, is equivalent to about 200 mcg of the free base of Compound 1. In a yet further embodiment, the amount present is equivalent to 200 mcg±50 mcg, or 200 mcg±45 mcg, or 200 mcg±40 mcg, or 200 mcg±35 mcg, or 200 mcg±30 mcg, or 200 mcg±25 mcg, or 200 mcg±20 mcg, or 200 mcg±15 mcg, or 200 mcg±10 mcg, or 200 mcg±5 mcg of the free base of Compound 1. In a yet further embodiment, the amount present is equivalent to an amount selected from 200 mcg, 195 mcg, 190 mcg, 185 mcg, 180 mg, 175 mcg, 170 mcg, 165 mcg, 160 mcg, 155 mcg and 150 mcg.

In yet another embodiment, the amount of Compound 1, or a pharmaceutically acceptable salt thereof, is equivalent to 200 mcg of the free base of Compound 1.

In one embodiment, Compound 1 may be administered by inhalation at a dose of about 150 mcg per day. In another embodiment, Compound 1 may be administered by inhalation at a dose of 150 mcg per day. In yet another embodiment Compound 1 may be administered by inhalation at a dose of about 300 mcg per day. In another embodiment, Compound 1 may be administered by inhalation at a dose of 300 mcg per day. In a further embodiment Compound 1 may be administered by inhalation at a dose of about 200 mcg per day. In another embodiment, Compound 1 may be administered by inhalation at a dose of 200 mcg per day. In general, Compound 1 will be administered as a once-daily dose.

In one embodiment, Compound 2 may be administered by inhalation at a dose of about 100 mcg per day. In another embodiment, Compound 2 may be administered by inhalation at a dose of 100 mcg per day. In yet another embodiment, Compound 2 may be administered by inhalation at a dose of about 200 mcg per day. In a further embodiment, Compound 2 may be administered by inhalation at a dose of 200 mcg per day. In general, Compound 2 will be administered as a once-daily dose.

In one embodiment, a succinate salt of Compound 1 provides the free base of Compound 1 in an amount of about 150 mcg. In another embodiment, a succinate salt of Compound 1 provides the free base of Compound 1 in an amount of 150 mcg. In another embodiment a succinate salt of Compound 1 provides the free base of Compound 1 in an amount of about 300 mcg. In another embodiment a succinate salt of Compound 1 provides the free base of Compound 1 in an amount of 300 mcg. In a further embodiment a succinate salt of Compound 1 provides the free base of Compound 1 in an amount of about 200 mcg. In another embodiment a succinate salt of Compound 1 provides the free base of Compound 1 in an amount of 200 mcg.

In a further embodiment, the present invention provides a pharmaceutical combination product for once-daily administration by inhalation, comprising Compound 1 at a dose of 150 mcg per day, and Compound 2 at a dose of 100 mcg per day.

In a further embodiment, the present invention provides a pharmaceutical combination product for once-daily administration by inhalation, comprising Compound 1 at a dose of 150 mcg per day, and Compound 2 at a dose of 200 mcg per day.

In a further embodiment, the present invention provides a pharmaceutical combination product for once-daily administration by inhalation, comprising Compound 1 at a dose of 300 mcg per day, and Compound 2 at a dose of 100 mcg per day.

In a further embodiment, the present invention provides a pharmaceutical combination product for once-daily administration by inhalation, comprising Compound 1 at a dose of 300 mcg per day, and Compound 2 at a dose of 200 mcg per day.

In a further embodiment, the present invention provides a pharmaceutical combination product for once-daily administration by inhalation, comprising Compound 1 at a dose of 200 mcg per day, and Compound 2 at a dose of 100 mcg per day.

In a further embodiment, the present invention provides a pharmaceutical combination product for once-daily administration by inhalation, comprising Compound 1 at a dose of 200 mcg per day, and Compound 2 at a dose of 200 mcg per day.

In one embodiment, Compound 1 may be administered by inhalation to deliver a respirable dose of about 40 mcg per day, for example a respirable dose of 40 mcg per day, for example from an inhalation dose of 150 mcg. In another embodiment, Compound 1 may be administered by inhalation to deliver a respirable dose of about 60 mcg per day, for example a respirable dose of 60 mcg per day, for example from an inhalation dose of 200 mcg. In a further embodiment, Compound 1 may be administered by inhalation to deliver a respirable dose of about 80 mcg per day, for example a respirable dose of 80 mcg per day, for example from an inhalation dose of 300 mcg.

As used herein, the term “respirable dose” means the fraction of the inhalation dose in which the particle size is less than 5 micrometres.

It will be appreciated that the individual compounds of the combination may be administered simultaneously, either in the same formulation or different pharmaceutical formulations, or sequentially. If there is sequential administration, the delay in administering the second active ingredient should not be such as to lose the benefit of any synergistic therapeutic effect of the combination of the active ingredients.

The individual compounds of the pharmaceutical combination product as described herein, Compound 1 and Compound 2, may thus be administered either i) simultaneously or sequentially in separate formulations, or ii) simultaneously in a combined pharmaceutical formulation or composition. Thus, Compound 1 and Compound 2 may be formulated separately and presented in separate packs or devices, or said individually formulated components may be presented in a single pack or device. Where appropriate, the individual compounds may be admixed within the same formulation, and presented as a fixed pharmaceutical combination. In general such formulations will include pharmaceutical carriers or excipients as described hereinafter, but combinations of the compounds without any carriers or excipients are also within the ambit of this invention. In one embodiment, the individual compounds of the pharmaceutical combination product may be administered simultaneously in a combined pharmaceutical formulation or composition. In another embodiment, Compound 1 and Compound 2 may be administered simultaneously or sequentially in separate pharmaceutical formulations or compositions. In a further embodiment, Compound 1 and Compound 2 are administered simultaneously in separate pharmaceutical formulations or compositions.

In further aspects the invention therefore provides: i) a pharmaceutical combination product comprising Compound 1 and Compound 2 presented separately for simultaneous or sequential administration; ii) a pharmaceutical combination product comprising Compound 1 and Compound 2 presented separately but held in the same pack or device, for simultaneous or sequential administration; and iii) a pharmaceutical combination product comprising Compound 1 and Compound 2, in a combined formulation (in admixture with each other) for simultaneous administration.

In each case, each of Compound 1 and/or Compound 2 may be formulated with or without pharmaceutical carriers or excipients.

The present invention further provides a pharmaceutical combination product comprising Compound 1 and Compound 2 wherein at least one of Compound 1 and Compound 2 is formulated with a pharmaceutically acceptable carrier or excipient.

The present invention further provides a pharmaceutical combination product comprising Compound 1 and Compound 2 wherein each of Compound 1 and Compound 2 is formulated with a pharmaceutically acceptable carrier or excipient.

In one embodiment of this invention compositions of Compounds 1 and 2 include those suitable for inhalation, including fine particle powders, or mists which may be generated and administered by means of various types of inhalers for example, reservoir dry powder inhalers, unit-dose dry powder inhalers, pre-metered multi-dose dry powder inhalers, nasal inhalers or pressurized metered dose inhalers, nebulisers or insufflators.

The compositions may be prepared by any of the methods well known in the art of pharmacy. In general, said methods include the step of bringing the active ingredient(s) into association with the carrier which constitutes one or more accessory ingredients. In general the compositions are prepared by uniformly and intimately bringing into association the active ingredient with liquid carriers or finely divided solid carriers or both and then, if necessary, shaping the product into the desired composition.

Powder compositions generally contain a powder mix for inhalation of the active ingredient and a suitable powder base (carrier/diluent/excipient substance) such as mono-, di or poly-saccharides (e.g. lactose or starch). Use of lactose is preferred. The lactose may be for example anhydrous lactose or α-lactose monohydrate. In one embodiment, the carrier is α-lactose monohydrate. Dry powder compositions may also include, in addition to the active ingredient and carrier, a further carrier or excipient (eg a ternary agent) such as a sugar ester, calcium stearate or magnesium stearate. Alternatively, the active ingredient may be presented without carriers or excipients. For the avoidance of doubt use of the term ‘composition’ or ‘formulation’ herein refers to the active ingredients either with or without excipients or carriers.

The compositions may be presented in unit dosage form. Dry powder compositions for topical delivery to the lung by inhalation may, for example, be presented in capsules and cartridges of for example gelatine, or blisters of for example laminated aluminium foil, for use in an inhaler or insufflator.

Each capsule, cartridge or blister may generally contain between 1-300 mcg of Compound 1 and/or between 1-200 mcg of Compound 2. Packaging of the formulation may be suitable for unit dose or multi-dose delivery. As indicated above Compound 1 and Compound 2 may be formulated independently or in admixture. Said compounds may thus be incorporated in separate unit doses or may be combined in a single unit dose with or without additional excipients as deemed necessary.

In a further embodiment, each capsule, cartridge or blister may contain 300 mcg of Compound 1 and/or 100 mcg of Compound 2.

In one embodiment, a composition suitable for inhaled administration may be incorporated into a plurality of sealed dose containers provided on medicament pack(s) mounted inside a suitable inhalation device. The containers may be rupturable, peelable or otherwise openable one-at-a-time and the doses of the dry powder composition administered by inhalation on a mouthpiece of the inhalation device, as known in the art. The medicament pack may take a number of different forms, for instance a disk-shape or an elongate strip. Representative inhalation devices are the DISKHALER™ and DISKUS™ devices, marketed by GlaxoSmithKline. The DISKUS™ inhalation device is, for example, described in GB 2242134A.

A dry powder inhalable composition may also be provided as a bulk reservoir in an inhalation device, the device then being provided with a metering mechanism for metering a dose of the composition from the reservoir to an inhalation channel where the metered dose is able to be inhaled by a patient inhaling at a mouthpiece of the device. Exemplary marketed devices of this type are TURBUHALER™ of AstraZeneca, TWISTHALER™ of Schering and CLICKHALER™ of Innovata.

A further delivery method for a dry powder inhalable composition is for metered doses of the composition to be provided in capsules (one dose per capsule) which are then loaded into an inhalation device, typically by the patient on demand. The device has means to rupture, pierce or otherwise open the capsule so that the dose is able to be entrained into the patient's lung when they inhale at the device mouthpiece. As marketed examples of such devices there may be mentioned ROTAHALER™ of GlaxoSmithKline and HANDIHALER™ of Boehringer Ingelheim.

Dry powder compositions may also be presented in another embodiment, the ELLIPTA™ inhalation device, which permits separate containment of two different components of the composition. Thus, for example, these components are administrable simultaneously but are stored separately, e.g. in separate pharmaceutical compositions, for example as described in WO 03/061743 A1 WO 2007/012871 A1 and/or WO2007/068896. In one embodiment, the ELLIPTA™ inhalation device has two peelable blister strips, each strip containing pre-metered doses in blister pockets arranged along its length, e.g., multiple containers within each blister strip. Said device has an internal indexing mechanism which, each time the device is actuated, peels opens a pocket of each strip and positions the blisters so that each newly exposed dose of each strip is adjacent to the manifold which communicates with the mouthpiece of the device. When the patient inhales at the mouthpiece, each dose is simultaneously drawn out of its associated pocket into the manifold and entrained via the mouthpiece into the patient's respiratory tract. A further device that permits separate containment of different components is DUOHALER™ of Innovata.

In addition, various structures of inhalation devices provide for the sequential delivery of the pharmaceutical composition(s) from the device, in addition to simultaneous delivery.

Other structures of dry powder inhalation devices provide for two components of the composition to be held together in a single capsule, cartridge or blister, wherein either each component is formulated separately, or both components are formulated together. The components are thus administered simultaneously.

In a further embodiment, the present invention provides Inhaler 1 wherein each composition is in unit dose form.

In a further embodiment, the present invention provides Inhaler 1 wherein the unit dose form is a capsule, cartridge or blister.

In a further embodiment, the present invention provides Inhaler 1 wherein Compound 1 is present in an amount of about 150 mcg to 300 mcg/dose, for example in an amount of 150 mcg to 300 mcg/dose, preferably a 150 mcg, 200 mcg or 300 mcg/dose.

In a further embodiment, the present invention provides Inhaler 1 wherein Compound 2 is present in an amount of about 100 mcg to 200 mcg/dose, for example in an amount of 100 mcg to 200 mcg/dose, preferably in an amount of 100 mcg or 200 mcg/dose.

Spray compositions for inhalation may, for example, be formulated as aqueous solutions or suspensions. The compositions may be delivered by means of a pressurised aerosol pack, such as a metered dose inhaler, with the use of a suitable liquefied propellant. Aerosol compositions suitable for inhalation can be either a suspension or a solution and generally contain the pharmaceutical product and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants e.g. oleic acid, lecithin or an oligolactic acid derivative e.g. as described in WO94/21229 and WO98/34596 and/or cosolvents e.g. ethanol. Pressurised formulations will generally be retained in a canister (e.g. an aluminium canister) dosed with a valve (e.g. a metering valve) and fitted into an actuator provided with a mouthpiece.

There is thus provided as a further aspect of the invention a pharmaceutical combination product comprising Compound 1, or a pharmaceutically acceptable salt thereof, wherein the free base of Compound 1 is provided in an amount of about 150 to about 300 mcg and about 100 to about 200 mcg of Compound 2, formulated individually or in admixture, with a fluorocarbon or hydrogen-containing chlorofluorocarbon as propellant, optionally in combination with a surface-active agent and/or a co-solvent. According to another aspect of the invention, the propellant is selected from 1,1,1,2-tetrafluoroethane, 1,1,1,2,3,3,3-heptafluoro-n-propane and mixtures thereof.

Another aspect of the invention is a pharmaceutical combination product consisting of Compound 1 and Compound 2 formulated individually or in admixture, with a fluorocarbon or hydrogen-containing chlorofluorocarbon as propellant, optionally in combination with a surface-active agent and/or a cosolvent. In another embodiment of the invention the propellant is selected from 1,1,1,2-tetrafluoroethane, or 1,1,1,2,3,3,3-heptafluoro-n-propane and mixtures thereof.

Where appropriate, compositions according to the invention may be buffered by the addition of suitable buffering agents.

Active ingredients for administration by inhalation desirably have a controlled particle size. The optimum particle size for inhalation into the bronchial system is usually 1-10 μm, preferably 2-5 μm. Particles having a size above 20 μm are generally too large when inhaled to reach the small airways. To achieve these particle sizes the particles of the active ingredient may be produced by size reduction using conventional means e.g. by micronisation. The desired fraction may be separated out by air classification or sieving. In one embodiment, the particles are crystalline. The particles may also be produced by alternative means such as spray drying, crystallisation or other particle engineering techniques.

The compounds of the invention may also be prepared as an amorphous molecular dispersion of drug substance in a polymer matrix such as HPMCAS (hydroxypropylmethylcellulose acetate succinate) using a process such as spray-dried dispersion (SDD). Such a technique is employed to improve properties such as stability and solubility.

Dry powder compositions according to the invention may comprise a carrier. The carrier when it is lactose e.g. α-lactose monohydrate, may form from about 91% to about 99%, e.g. 97.7-99.0% or 91.0-99.2% by weight of the formulation. In general, the particle size of the carrier, for example lactose, will be much greater than the inhaled medicament within the present invention. When the carrier is lactose it will typically be present as milled lactose, having a MMD (mass median diameter) of 60-90 μm.

The lactose component may comprise a fine lactose fraction. The ‘fine’ lactose fraction is defined as the fraction of lactose having a particle size of less than 7 μm, such as less than 6 μm, for example less than 5 μm. The particle size of the ‘fine’ lactose fraction may be less than 4.5 μm. The fine lactose fraction, if present, may comprise 2 to 10% by weight of the total lactose component, such as 3 to 6% by weight fine lactose, for example 4.5% by weight fine lactose.

Magnesium stearate, if present in the composition, is generally used in an amount of about 0.2% to about 2%, e.g. 0.6 to 2% or 0.5 to 1.75%, e.g. 0.6%, 0.75%, 1%, 1.25% or 1.5% w/w, based on the total weight of the composition. The magnesium stearate will typically have a particle size in the range 1 to 50 μm, and more particularly 1-20 μm, e.g. 1-10 μm. Commercial sources of magnesium stearate include Peter Greven, Covidien/Mallinckodt and FACI. In one embodiment, the composition does not comprise magnesium stearate.

Intranasal sprays may be formulated with aqueous or non-aqueous vehicles with the addition of agents such as thickening agents, buffer salts or acid or alkali to adjust the pH, isotonicity adjusting agents or anti-oxidants.

Solutions for inhalation by nebulisation may be formulated with an aqueous vehicle with the addition of agents such as acid or alkali, buffer salts, isotonicity adjusting agents or antimicrobials. They may be sterilized by filtration or heating in an autoclave, or presented as a non-sterile product.

The invention also provides a method of preparing a pharmaceutical combination product as defined herein, the method comprising either:

(a) preparing separate pharmaceutical compositions for administration of the individual compounds of the combination either simultaneously or sequentially, or
(b) preparing a combined pharmaceutical composition for administration of the individual compounds together in the combination for simultaneous use, wherein the pharmaceutical composition comprises the combination together with one or more pharmaceutically acceptable carriers and/or excipients.

Compound 1 may be prepared as described in WO 2007/090859 which is incorporated by reference herein.

Compound 2 (fluticasone furoate), also referred to as 6α,9α-difluoro-17α-[(2-furanylcarbonyl)oxy]-11β-hydroxy-16α-methyl-3-oxo-androsta-1,4-diene-17β-carbothioic acid S-fluoromethyl ester, is described as Example 1 in WO02/12265 (Glaxo Group Limited), which is incorporated by reference herein.

### Embodiments of the Invention

In a first aspect of the invention, there is provided a pharmaceutical combination comprising:

a) Compound 1, biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester,

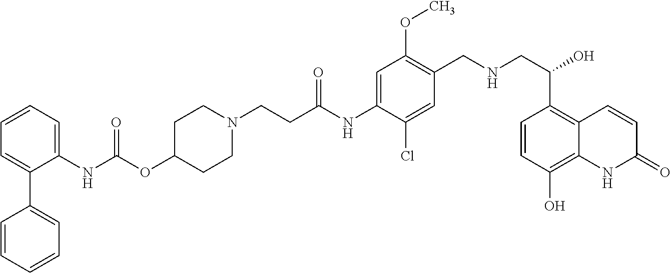 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, which is provided in an amount equivalent to: about 150 to about 300 mcg of the free base of Compound 1
and
b) about 100 to about 200 mcg of Compound 2, fluticasone furoate

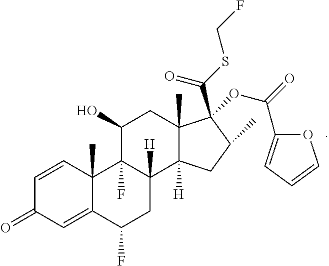

In one embodiment, Compound 1 is provided in the form of a succinate salt.

In one embodiment, there is provided a pharmaceutical combination comprising:

a) Compound 1, biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester,

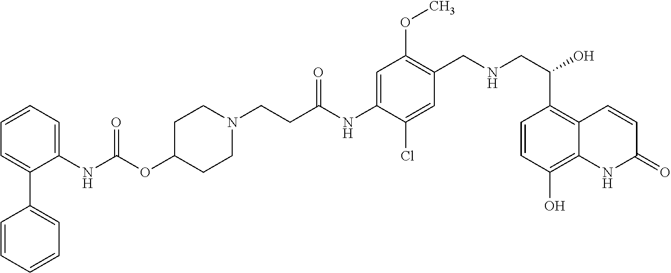 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, which is provided in an amount equivalent to 150 to 300 mcg of the free base of Compound 1
and
b) 100 to 200 mcg of Compound 2, fluticasone furoate

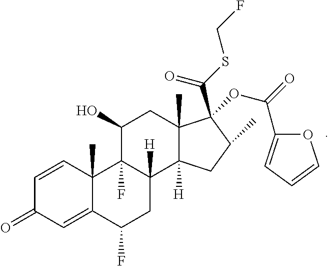

In one embodiment, Compound 1 is provided in the form of a succinate salt.

In one embodiment, the free base equivalent of Compound 1 is in an amount of about 150 mcg and Compound 2 is in an amount of about 100 mcg. In another embodiment, the free base equivalent of Compound 1 is in an amount of 150 mcg and Compound 2 is in an amount of 100 mcg.

In one embodiment, the free base equivalent of Compound 1 is in an amount of about 200 mcg and Compound 2 is in an amount of about 100 mcg. In another embodiment, the free base equivalent of Compound 1 is in an amount of 200 mcg and Compound 2 is in an amount of 100 mcg.

In one embodiment, the free base equivalent of Compound 1 is in an amount of about 300 mcg and Compound 2 is in an amount of about 100 mcg. In another embodiment, the free base equivalent of Compound 1 is in an amount of 300 mcg and Compound 2 is in an amount of 100 mcg.

In one embodiment, the free base equivalent of Compound 1 is in an amount of about 150 mcg and Compound 2 is in an amount of about 200 mcg. In one embodiment, the free base equivalent of Compound 1 is in an amount of 150 mcg and Compound 2 is in an amount of 200 mcg.

In one embodiment, the free base equivalent of Compound 1 is in an amount of about 200 mcg and Compound 2 is in an amount of about 200 mcg. In another embodiment, the free base equivalent of Compound 1 is in an amount of 200 mcg and Compound 2 is in an amount of 200 mcg.

In one embodiment, the free base equivalent of Compound 1 is in an amount of about 300 mcg and Compound 2 is in an amount of about 200 mcg. In another embodiment, the free base equivalent of Compound 1 is in an amount of 300 mcg and Compound 2 is in an amount of 200 mcg.

In one embodiment, Compound 1, or a pharmaceutically acceptable salt thereof, and Compound 2 are presented in separate formulations adapted for simultaneous or sequential administration.

In one embodiment, there is provided a composition comprising a pharmaceutical combination as defined hereinabove, wherein at least one of Compound 1 and Compound 2 is formulated with a pharmaceutically acceptable carrier or excipient.

In one embodiment the pharmaceutical combination as defined hereinabove is in a form suitable for administration by oral or nasal inhalation. In another embodiment, the form is suitable for administration by inhalation via a medicament dispenser selected from a reservoir dry powder inhaler, a unit-dose dry powder inhaler, a pre-metered multi-dose dry powder inhaler, a nasal inhaler or a pressurized metered dose inhaler. In a further embodiment, each of Compound 1 and Compound 2 is presented in the form of a dry powder composition. In another embodiment, Compound 1 and Compound 2 are presented as separate compositions. In a further embodiment, at least one of said compositions of Compound 1 or Compound 2 contains a carrier or excipient. In one embodiment, the carrier is lactose. In another embodiment, the said separate compositions are in unit dose form. In one embodiment, the unit dose form is in a capsule, cartridge or blister pack. In one embodiment, the composition is administered via a dry powder inhaler. In one embodiment, there is provided a dry powder inhaler containing a pharmaceutical combination as defined hereinabove.

In one embodiment, there is provided a pharmaceutical combination as defined hereinabove, for use in therapy.

In one embodiment, there is provided a pharmaceutical combination as defined hereinabove, for use in the treatment of a pulmonary disorder. In another embodiment, the pulmonary disorder is COPD, asthma or ACOS.

In one embodiment, there is provided the use of the pharmaceutical combination as defined hereinabove, in the manufacture of a medicament for the treatment of a pulmonary disorder. In one embodiment, Compound 1 and Compound 2 are given by simultaneous or sequential administration, in any order. In one embodiment, the pulmonary disorder is COPD, asthma or ACOS.

In one embodiment, there is provided a method for the treatment of a pulmonary disorder, comprising administering to a patient in need thereof, a pharmaceutical combination as defined hereinabove. In another embodiment, in said method the active ingredients of said pharmaceutical combination are administered either simultaneously or sequentially. In a further embodiment, in said method the disease is selected from the group consisting of COPD, chronic bronchitis, asthma, chronic respiratory obstruction, pulmonary emphysema, allergic rhinitis, small airways disease, bronchiectasis, asthma, ACOS and cystic fibrosis. In one embodiment, in said method the disease is COPD.

In a second aspect the present invention provides a pharmaceutical combination product comprising:

a) Compound 1, biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester

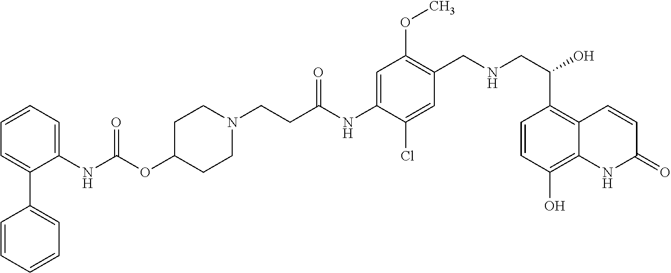 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]

or a pharmaceutically acceptable salt thereof, wherein the free base of Compound 1 is provided in an amount of 150 mcg to 300 mcg
and
b) 100 mcg to 200 mcg of Compound 2, fluticasone furoate

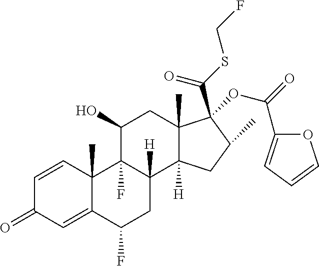

for the treatment of inflammatory or respiratory disorders, such as COPD and asthma.

In one embodiment, Compound 1 is in an amount of 150 mcg and Compound 2 is in an amount of 100 mcg. In another embodiment, Compound 1 is in an amount of 300 mcg and Compound 2 is in an amount of 100 mcg. In a further embodiment, Compound 1 is in an amount of 150 mcg and Compound 2 is in an amount of 200 mcg. In another embodiment, Compound 1 is in an amount of 300 mcg and Compound 2 is in an amount of 200 mcg. In a yet further embodiment, a succinate salt of Compound 1 provides the free base of Compound 1 in an amount of 150 to 300 mcg.

In one embodiment, Compound 1 and Compound 2 are presented in a form adapted for separate, sequential or simultaneous administration.

In one embodiment, at least one of Compound 1 and Compound 2 is formulated with a pharmaceutically acceptable carrier or excipient.

In one embodiment, the pharmaceutical combination product is in a form suitable for administration by oral or nasal inhalation. In a further embodiment, the form is suitable for administration by inhalation via a medicament dispenser selected from a reservoir dry powder inhaler, a unit-dose dry powder inhaler, a pre-metered multi-dose dry powder inhaler, a nasal inhaler or a pressurized metered dose inhaler. In a yet further embodiment, each of Compound 1 and Compound 2 is presented in the form of a dry powder composition. In another embodiment, Compound 1 and Compound 2 are presented as separate or admixed compositions. In a further embodiment, at least one of said compositions of Compound 1 or Compound 2 contains a carrier. In another embodiment, the carrier is lactose. In another embodiment, said separate or admixed compositions are in unit dose form. In a further embodiment, the unit dose form is in a capsule, cartridge or blister pack. In a yet further embodiment, the composition is administered via a dry powder inhaler. In another embodiment, there is provided a dry powder inhaler containing a product as defined hereinabove.

In one embodiment there is provided the use of the pharmaceutical combination product as hereinabove defined in the manufacture of a medicament for the prophylaxis or treatment of conditions for which administration of one or more of Compound 1 and Compound 2 is indicated. In a further embodiment, the use is for the treatment of inflammatory or respiratory tract diseases, by simultaneous or sequential administration, in any order, of Compound 1 and Compound 2. In another embodiment, the use is for the manufacture of a medicament for the treatment of chronic obstructive pulmonary disease (COPD) and/or asthma by simultaneous or sequential administration of Compound 1 and Compound 2.

In one embodiment there is provided a method for the prophylaxis or treatment of inflammatory or respiratory tract diseases, comprising administering to a patient in need thereof, the pharmaceutical combination product as hereinabove defined. In another embodiment, the active ingredients of said product are administered either sequentially or simultaneously in said method. In a further embodiment, in said method, the disease is selected from the group consisting of chronic obstructive lung disease, chronic bronchitis, asthma, chronic respiratory obstruction, pulmonary fibrosis, pulmonary emphysema, allergic rhinitis, small airways disease, bronchiectasis, asthma and chronic obstructive pulmonary disease overlap syndrome (ACOS), and cystic fibrosis. In a yet further embodiment, in said method the disease is chronic obstructive lung disease (COPD).

### Clinical Studies

### Single Compound Therapy

### Compound 1

In a randomised, double-blind, double-dummy, placebo and active comparator controlled, cross-over study designed to evaluate the safety, tolerability, efficacy, systemic pharmacokinetics and pharmacodynamics of Compound 1 in subjects with COPD, two once-daily doses (400 mcg and 1200 mcg DISKUS), taken over a 14-day treatment period, produced statistically significant improvements in pulmonary function compared to placebo (Bateman E D et al., Pharmacodynamics of GSK961081, a bi-functional molecule, in patients with COPD. Pulmonary Pharmacology & Therapeutics 2013). Compound 1 demonstrated sustained bronchodilation similar to tiotropium once-daily (18 mcg) plus salmeterol twice-daily (50 mcg), but with a more rapid onset, and was well tolerated.

A further, randomised, double-blind, double-dummy, placebo and salmeterol controlled parallel group study evaluated the efficacy and safety, of three once-daily (100 mcg, 400 mcg and 800 mcg DISKUS) and three twice-daily (100 mcg, 200 mcg and 400 mcg DISKUS) doses of Compound 1 in subjects with COPD over a 28-day treatment period (Wielders et al, A new class of bronchodilator improves lung function in COPD: a trial with GSK961081; European Respiratory Journal 2013). Compound 1 produced statistically and clinically significant differences from placebo at all doses investigated for trough FEV1 on day 29. Compound 1 had a rapid onset, showed no significant differences between once-daily and twice-daily dosing, and was well tolerated.

### Compound 2

### Asthma

Several clinical pharmacology studies have been conducted using fluticasone furoate to investigate the safety and efficacy of this compound in asthmatic patients.

In one such study, the safety and efficacy of four doses of fluticasone furoate in subjects with persistent uncontrolled asthma were evaluated. In this study, which was a randomised, double-blind, placebo-controlled, parallel group study, 598 patients received one of six treatments: fluticasone furoate (25, 50, 100 or 200 mcg) once daily, fluticasone propionate 100 mcg twice daily or placebo for 8 weeks. The primary endpoint was change from baseline in trough (pre-dose) forced expiratory volume in 1 second (FEV₁) at Week 8. At Week 8, relative to placebo fluticasone furoate 50-200 mcg once daily had significantly greater increases in trough FEV₁ from baseline (p<0.05*) with fluticasone furoate 100 mcg and 200 mcg achieving a >200 mL increase. This study supports the use of fluticasone furoate (100 or 200 mcg once-daily) for the treatment of persistent uncontrolled asthma.

### COPD

In contrast to patients with asthma, lung function is minimally impacted by inhaled corticosteroids in patients with COPD. Replicate studies clinical studies were designed to evaluate the efficacy and safety of the three doses of fluticasone furoate (50, 100 or 200 mcg) when combined with the bronchodilator, vilanterol 25 mcg once daily versus vilanterol alone over a 1-year period. With respect to efficacy, these studies were designed to evaluate the benefit of fluticasone furoate in the combination, in decreasing the annual rate of moderate and severe exacerbations in patients with COPD with an exacerbation history. The pooled analysis from these replicate studies demonstrated that all three doses of fluticasone furoate when combined with vilanterol provided significantly (p≤0.014*) greater reductions in the LS (least squares) mean annual rate of moderate or severe COPD exacerbations compared with vilanterol treatment alone. The fluticasone furoate (100 mcg) and vilanterol (25 mcg) combination product demonstrated the greatest reduction as compared with vilaneterol alone (27%; p<0.001*), with no incremental efficacy benefit achieved with the higher dose fluticasone furoate (200 mcg) and vilanterol (25 mcg) combination product as compared with vilanterol alone (23%; p<0.001*). *“p” is a statistical notation indicating the probability that the observation made is due to chance alone.

### Combination Therapy

A combination of Compound 1 and Compound 2 has been administered to 48 healthy volunteers, aged 18 to 57 years, as part of a clinical trial to assess the in vivo drug delivery, systemic pharmacokinetics, safety and tolerability of Compound 1 and Compound 2 when administered as a combination product via the ELLIPTA inhaler in comparison to Compound 1 alone (using the ELLIPTA inhaler in dual and single strip configurations) or Compound 2 alone (using the ELLIPTA inhaler in dual strip configuration). The study was an open-label, randomised, six-way cross-over study wherein subjects received a single dose of:

Compound 1 1200 mcg (DISKUS),

Compound 1 1200 mcg (ELLIPTA inhaler single strip configuration),

Compound 1 1200 mcg (ELLIPTA inhaler dual strip configuration),

Compound 1 1200 mcg/Compound 2 300 mcg (ELLIPTA inhaler),

Compound 1 900 mcg/Compound 2 300 mcg (ELLIPTA inhaler), or

Compound 2 300 mcg (ELLIPTA dual strip configuration)

at each of the six treatment periods.

Single inhaled doses and the combination were found to be well tolerated.

### Pharmaceutical Formulations

### Example Perorations of Compound 1 Succinate Blend

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns).

An appropriate quantity of micronised Compound 1 succinate is then blended with the sieved α-lactose monohydrate using either a high shear mixer (a QMM, PMA or TRV series mixer, such as TRV25 or TRV65) or a low shear tumbling blender (a Turbula mixer).

### Representative Batch Formula for Compound 1 Succinate Powder Blend (150 Microgram Per Blister)

| Ingredient | Quantity | |
|---|---|---|
| Micronised Compound 1 succinate | 167 | g |
| Lactose Monohydrate | To 12.0 | kg |

Note:
167 g of Compound 1 succinate is equivalent to 144 g of the free base. The quantity of Compound 1 succinate added may be adjusted to reflect the assigned purity of the input drug substance.

### Representative Batch Formula for Compound 1 Succinate Powder Blend (300 Microgram Per Blister)

| Ingredient | Quantity | |
|---|---|---|
| Micronised Compound 1 succinate | 334 | g |
| Lactose Monohydrate | To 12.0 | kg |

Note:
334 g of Compound 1 succinate is equivalent to 288 g of the free base. The quantity of Compound 1 succinate added may be adjusted to reflect the assigned purity of the input drug substance.

### Example Blending Parameters (Using a TRV25, 12 kg Scale, Compound 1 Succinate Powder Blend (150 or 300 Microgram Blister)

| Time (mins) | Approximate Speed (rpm) |
|---|---|
| 11 | 500 |

### Example Preparations of Compound 2 Blend

Pharmaceutical grade α-lactose monohydrate, sourced from DMV Fronterra Excipients, complying with the requirements of Ph.Eur/USNF may be used. Before use, the α-lactose monohydrate may be sieved through a coarse screen (for example with a mesh size 500 or 800 microns).

An appropriate quantity of micronised Compound 2 is then blended with the sieved α-lactose monohydrate using either a high shear mixer (a QMM, PMA or TRV series mixer, such as TRV25 or TRV65) or a low shear tumbling blender (a Turbula mixer).

### Representative Batch Formula for Compound 2 Powder Blend (100 Microgram Per Blister

| Ingredient | Quantity | |
|---|---|---|
| Micronised Compound 2 | 84 | g |
| Lactose Monohydrate | To 10.5 | kg |

Note:
The quantity of Compound 2 added may be adjusted to reflect the assigned purity of the input drug substance.

### Representative Batch Formula for Compound 2 Powder Blend (200 Microgram Per Blister

| Ingredient | Quantity | |
|---|---|---|
| Micronised Compound 2 | 168 | g |
| Lactose Monohydrate | To 10.5 | kg |

Note:
The quantity of Compound 2 added may be adjusted to reflect the assigned purity of the input drug substance.

### Example Blending Parameters (Using a TRV25, 10.5 Kg Scale, Compound 2 Powder Blend (100 or 200 Microgram Blister)

| Time (mins) | Approximate Speed (rpm) |
|---|---|
| 7 | 550 |

### Example Blister Strip Preparation

The blended compositions of Compound 1 and Compound 2 may then be transferred into separate blister strips (typical nominal mean quantity of blend per blister is 12.5-13.5 mg) of the type generally used for the supply of dry powder for inhalation and the blister strips sealed in the customary fashion.

### Example Dry Powder Inhaler Devices

Compound 1 succinate and Compound 2 as an inhalation powder may be administered in a DPI device containing two blister strips. One strip contains a blend of micronised Compound 1 succinate (approximately 150 or 300 micrograms per blister) and lactose monohydrate. The second strip contains a blend of micronised Compound 2 (approximately 100 or 200 micrograms per blister) and lactose monohydrate. The DPI device will deliver, when actuated, the contents of a single blister simultaneously from each of the two blister strips. Each blister strip is a double foil laminate containing up to 30 filled blisters per strip.

### Using the Above-Described Procedure the Following Exemplary Formulations May be Prepared:

| | Mass of | | |
|---|---|---|---|
| | compound 1 | Mass of | Total quantity |
| | succinate | compound 2 | of α-lactose |
| Strip | (micronised) | (micronised) | monohydrate blend |
| Number | per blister | per blister | per blister |
| 1 | 174 or 348 mcg | — | 13 mg |
| 2 | — | 100 or 200 mcg | 13 mg |

Note:
174 mcg of Compound 1 succinate is equivalent to 150 mcg of the free base and 348 mcg of Compound 1 succinate is equivalent to 300 mcg of the free base. The quantity of Compound 1 succinate or Compound 2 added may be adjusted to reflect the assigned purity of the input drug substance.

All publications, including but not limited to patents and patent applications, cited in this specification are herein incorporated by reference as if each individual publication were specifically and individually indicated to be incorporated by reference herein as though fully set forth.

The above description fully discloses the invention including preferred embodiments thereof. Modifications and improvements of the embodiments specifically disclosed herein are within the scope of the following claims. Without further elaboration, it is believed that one skilled in the art can, using the preceding description, utilize the present invention to its fullest extent. Therefore, the Examples herein are to be construed as merely illustrative and not a limitation of the scope of the present invention in any way. The embodiments of the invention in which an exclusive property or privilege is claimed are defined as follows.

What is claimed is:

1. A pharmaceutical combination comprising:
a) Compound 1, biphenyl-2-ylcarbamic acid 1-[2-(2-chloro-4-{[(R)-2-hydroxy-2-(8-hydroxy-2-oxo-1,2-dihydroquinolin-5-yl)ethylamino]methyl}-5-methoxyphenylcarbamoyl)ethyl]piperidin-4-yl ester, 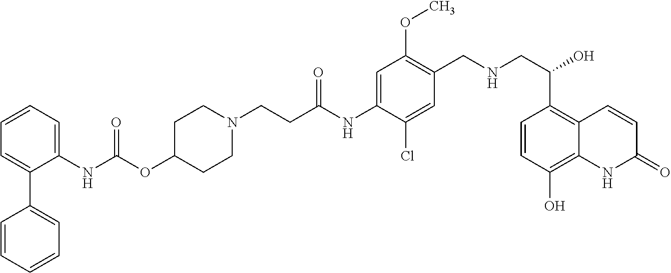 [SMILES: COc1cc(NC(=O)CCN2CCC(OC(=O)Nc3ccccc3-c3ccccc3)CC2)c(Cl)cc1CNC[C@H](O)c1ccc(O)c2[nH]c(=O)ccc12]
or a pharmaceutically acceptable salt thereof, which is provided in an amount equivalent to: about 150 to about 300 mcg of the free base of Compound 1
and
b) about 100 to about 200 mcg of Compound 2, fluticasone furoate

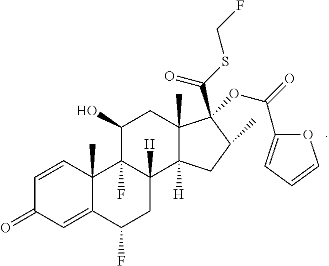

2. A pharmaceutical combination according to claim 1, wherein Compound 1 is provided in the form of a succinate salt.

3. A pharmaceutical combination according to claim 1, wherein the free base equivalent of Compound 1 is in an amount of about 300 mcg and Compound 2 is in an amount of about 100 mcg.

4. A pharmaceutical combination according to claim 1, wherein Compound 1, or a pharmaceutically acceptable salt thereof, and Compound 2 are presented in separate formulations adapted for simultaneous or sequential administration.

5. A pharmaceutical composition comprising a pharmaceutical combination according to claim 1, wherein at least one of Compound 1 and Compound 2 is formulated with a pharmaceutically acceptable carrier or excipient.

6. A pharmaceutical combination according to claim 1 in a form suitable for administration by oral or nasal inhalation.

7. A pharmaceutical combination according to claim 6 wherein each of Compound 1 and Compound 2 is presented in the form of a dry powder composition.

8. A pharmaceutical combination according to claim 7 wherein Compound 1 and Compound 2 are presented as separate compositions.

9. A pharmaceutical combination according to claim 7 wherein at least one of said compositions of Compound 1 or Compound 2 contains a carrier or excipient.

10. A dry powder inhaler comprising a pharmaceutical combination according to claim 1.

11-14. (canceled)

15. A method for the treatment of a pulmonary disorder, comprising administering to a patient in need thereof, a pharmaceutical combination according to claim 1.

16. A method according to claim 15, wherein the pulmonary disorder is COPD, asthma or ACOS
